(19) United States
(12) Patent Application Publication
(10) Pub. No.: US 2018/0228930 A1
(43) Pub. Date: Aug. 16, 2018

(54) WICKLESS FRAGRANCE DELIVERY METHODS AND SYSTEMS

(71) Applicant: Aromair Fine Fragrance Company, New Albany, OH (US)

(72) Inventors: Jason Paul Davis, Johnstown, OH (US); Irina Morosov, Columbus, OH (US)

(21) Appl. No.: 15/893,445

(22) Filed: Feb. 9, 2018

Related U.S. Application Data
(60) Provisional application No. 62/459,470, filed on Feb. 15, 2017.

(51) Int. Cl. A61L 9/03

(52) U.S. Cl. CPC A61L 9/03; A61L 9/035; A61L 9/032

(57) ABSTRACT

Systems and methods herein provide wickless fragrance delivery. In embodiments, fragrance aroma chemicals are delivered from a fragrance oil containing pod or cartridge by applying direct heat to said pod. Upon a particular temperature (or temperature range) being reached, the pod releases fragrance at a desired intensity. The heated pod releases fragrance at a cooler temperature as compared to traditional wax systems, traditional plug-in devices, and/or traditional diffusers. As such, the heater devices of the systems disclosed herein operate at a cooler temperature as compared to traditional fragrance systems. The comparatively cooler temperature makes the devices disclosed herein more energy efficient than traditional fragrance systems. In addition, the pods themselves are more efficient and utilize less fragrance as compared to traditional fragrance systems. Moreover, this device is designed to cycle warmer/cooler temperatures at programmed times. This temperature cycle fluctuates the amount of fragrance released, preventing the oversaturation of fragrance into the environment. This prevention of oversaturation is crucial for preventing human anosmia (e.g., the fragrance is no longer noticeable to the user due to overexposure).

### CROSS-REFERENCE TO RELATED APPLICATIONS

This application claims the benefit of U.S. Provisional Patent Application No. 62/459,470, entitled “Wickless Fragrance Delivery Methods and Systems” filed Feb. 15, 2017, the disclosure of which is hereby incorporated herein by reference.

### BRIEF SUMMARY OF THE INVENTION

Systems and methods herein provide wickless fragrance delivery. In embodiments, one or more fragrance is delivered from a fragrance pod having aroma chemicals therein. The fragrance pods herein passively release fragrance (by way of diffusion), and as such need no heat to release the aroma chemicals of the fragrance pod. That being said, if desired, the fragrance pods are designed such that a low amount of heat may be used assist the release of the aroma chemicals of the fragrance pod. Systems disclosed herein use fragrance pods having an first driving force (e.g., passive diffusion) behind the aroma release and a secondary driving force (e.g., low temperature heating) behind the fragrance release. Because the systems described herein utilize two different driving forces, more fragrance is released from the fragrance pod than would be released from pods using only a single driving force. As a result, the environmentally conscience fragrance pod contains less non-volatile compounds at the end of life, thereby causing less aroma chemicals to be discarded in landfills as compared to traditional systems. Further, because more of the aroma chemicals are used up during the life of the fragrance pod, the fragrance pod itself is more efficient because it may be infused with up to 60 percent less aroma chemicals as compared to traditional systems, while producing the same aroma affect.

Additionally, the fragrance pod is specifically designed to be driven by passive diffusion and heat; as such, with heat is added to assist the release of the aroma chemical, the secondary driving effect may be achieved with the use a very little heat. Accordingly, the wickless fragrance delivery systems disclosed herein are more efficient than traditional heat driven systems because the system described herein produce the same aroma affect with less heat and less electricity.

The fragrance pods herein, which are designed to be driven by a first driving force (e.g., passive diffusion), as well as a secondary driving force (e.g., heat), allows for the use of a more diverse combination of aroma chemicals, as compared to traditional fragrance pods. Typically aroma chemicals are designed to fit the method of transport, but in the present systems which use a passively diffusing substrate in combination with low heat, it creates a whole new palate of aroma chemical combinations. For example, vanilla is a very difficult aroma chemical to use with a passive substrate (e.g., membrane) because the molecules are very large. With heat, however, a larger palate of vanillas, which previously were unavailable for passive substrates are now available for use. As such, using low heat as a second driving force allows for a more diverse aroma palate. Further, using the second driving force allows for the reduction of solvents typically used to facilitate passive fragrance release. For example, solvents (e.g., hydrocarbons) are traditionally added to typical membrane fragrances to aid the transport (e.g., open the pores of the membrane) thereby causing diffusion to occur more quickly and allow larger molecules to pass through. In these traditional systems, when the hydrocarbons are gone, the membrane pores close, and any aroma chemicals remaining in the membrane are trapped and no longer diffuse aroma. Further, when larger molecules are passing through, the mere reducing of hydrocarbons cause the pores' size to shrink, causing the pores to clog and trap the remaining aroma chemicals to be trapped therein. In the systems described herein, due to using a second driving force (e.g., low heat) the traditional amounts of hydrocarbons are not necessary. The removal of excess hydrocarbons minimizes chemical exposure to the user and resolve the problems caused when hydrocarbons are expended. This advantage makes the fragrance pods utilize much more of the aroma chemicals therein making the fragrance pods more environmentally friendly and efficient.

Regarding traditional heating systems, a user is likely to come into contact with the aroma chemicals (e.g., wax, oils, etc.), which can not only be hot and toxic, but may also stain carpet, clothing, and skin. The aroma chemicals of the present systems are contained within a fragrance pod, so the user never has to touch the aroma chemicals. Further, the aroma chemicals are released at a much lower temperature, so the threat of burns is diminished. Moreover, the fragrance pod is clean and easy to replace without risk to carpet, skin, etc.

Further, wickless fragrance systems herein may be double fused and some systems may include an electrical currency switch allowing a user to select between two of more voltage inputs. For example, a user may choose between 240v and 110v. Further still, the wickless fragrance systems do not utilize a wick. As such, fire, which is a known hazard to child, pets, and the like, is not used as a heating mechanism.

As a result, example wickless fragrance systems disclosed herein are as aromatically effective as traditional systems, but the systems described herein utilize less energy thereby conserving electricity and prolonging the life of batteries, operate at lower temperature without the use of fire making the system safer, and are more environmentally friendly than traditional fragrance systems by reducing human contact with chemicals and reducing the amount of chemicals and batteries that are discarded in landfills.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWING

For a more complete understanding of the present invention, reference is now made to the following descriptions taken in conjunction with the accompanying drawing, in which:

FIG. 1A illustrates an example wickless fragrance system;

FIG. 1B illustrates circuitry of an example wickless fragrance system;

FIG. 1C illustrates circuitry of an example wickless fragrance system;

FIG. 1D illustrates an example method of using an example wickless fragrance system;

FIG. 1E illustrates an example time verses temperature graph of an example wickless fragrance system;

FIG. 1F illustrates an example method of using an example wickless fragrance system;

FIG. 1G illustrates an example method of using an example wickless fragrance system;

FIG. 2A illustrates an example wickless fragrance system;

FIG. 2B illustrates an example wickless fragrance system;

FIG. 2C illustrates circuitry of an example wickless fragrance system;

FIG. 2D illustrates circuitry of an example wickless fragrance system;

FIG. 2E illustrates an example method of using an example wickless fragrance system;

FIG. 2F illustrates an example method of using an example wickless fragrance system;

FIG. 2G illustrates an example time verses temperature graph of an example wickless fragrance system;

FIG. 3A illustrates an example wickless fragrance system;

FIG. 3B illustrates circuitry of an example wickless fragrance system;

FIG. 3C illustrates circuitry of an example wickless fragrance system;

FIG. 4A illustrates an example wickless fragrance system;

FIG. 4B illustrates an example wickless fragrance system; and

FIG. 4C illustrates an example method of using an example wickless fragrance system;

FIG. 4D illustrates an example method of using an example wickless fragrance system;

FIG. 4E illustrates an example time verses temperature graph of an example wickless fragrance system;

FIG. 5A illustrates an example wickless fragrance system; and

FIG. 5B illustrates an example wickless fragrance system.

### DETAILED DESCRIPTION OF THE INVENTION

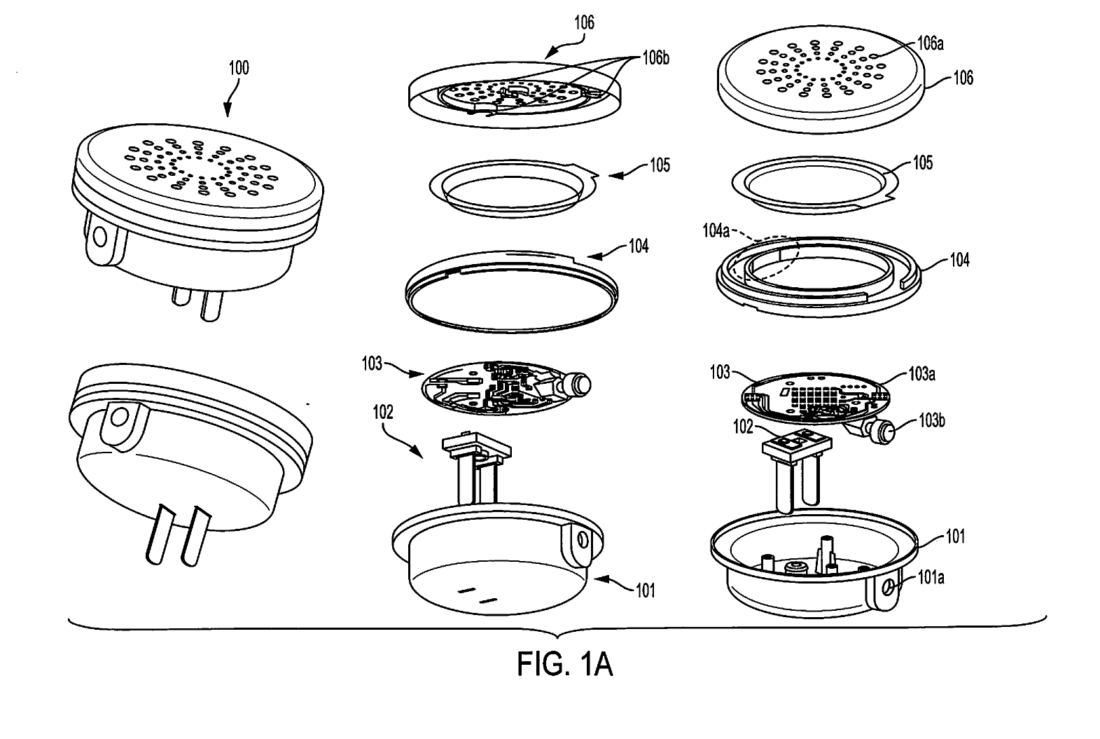

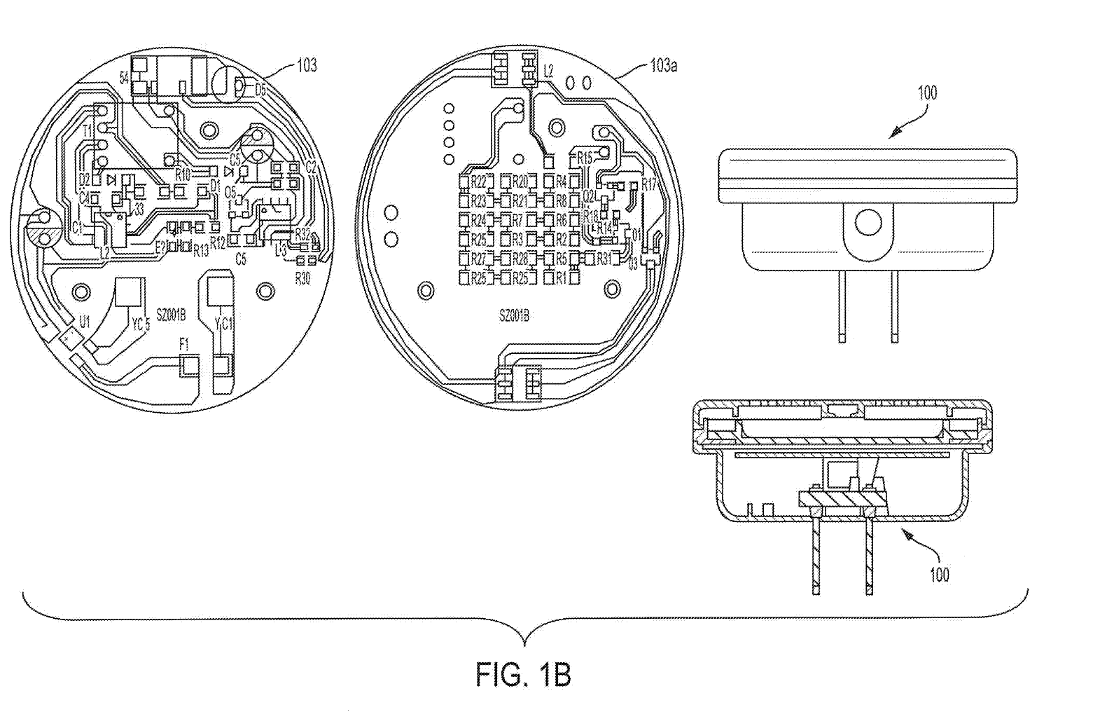

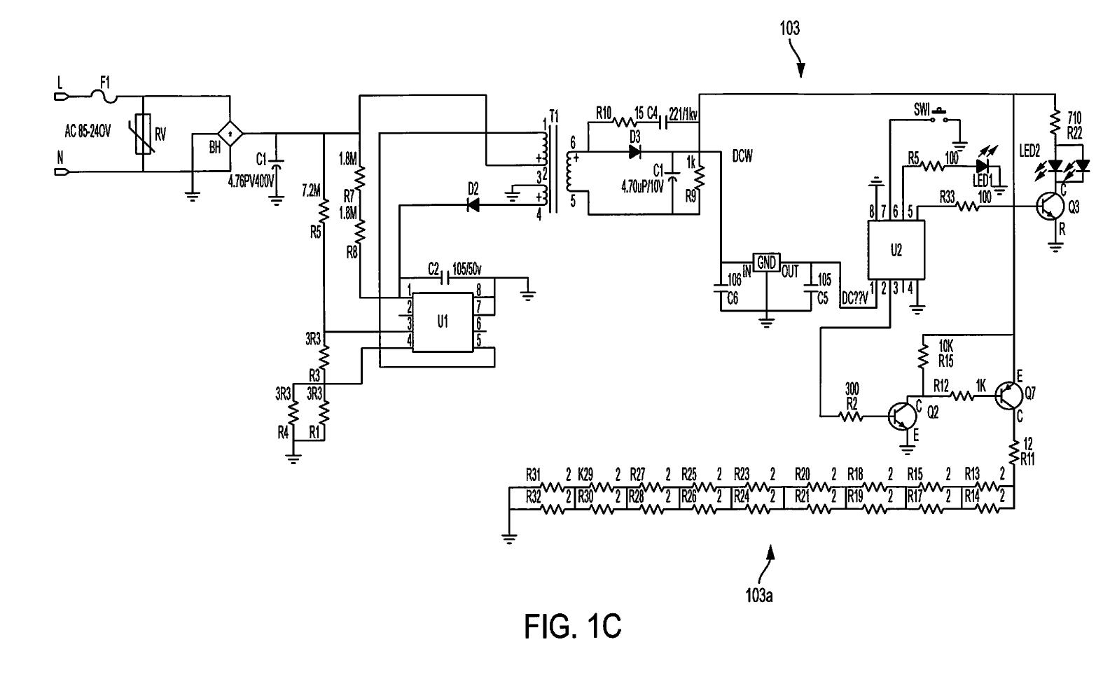

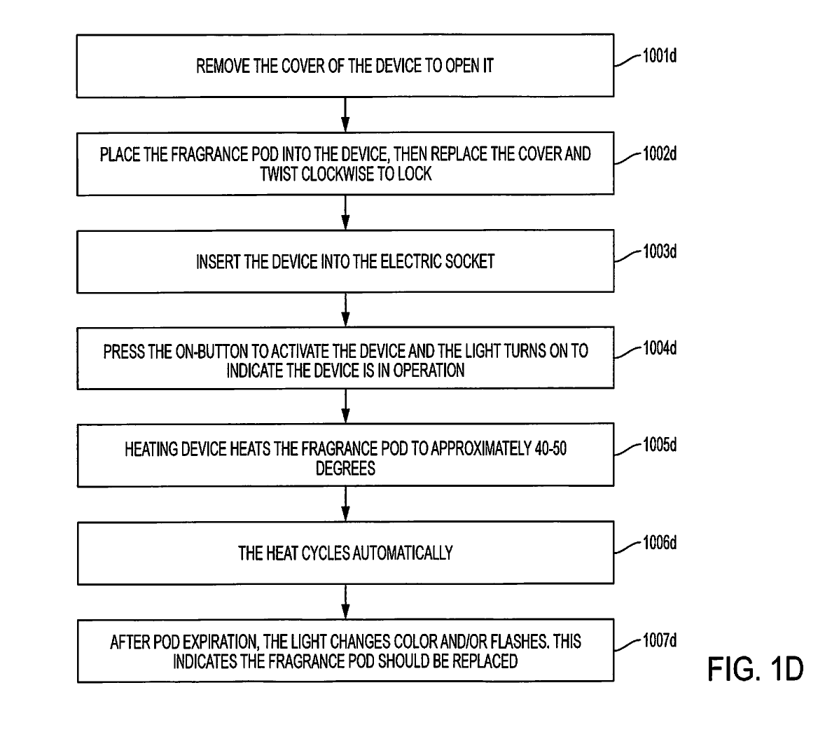

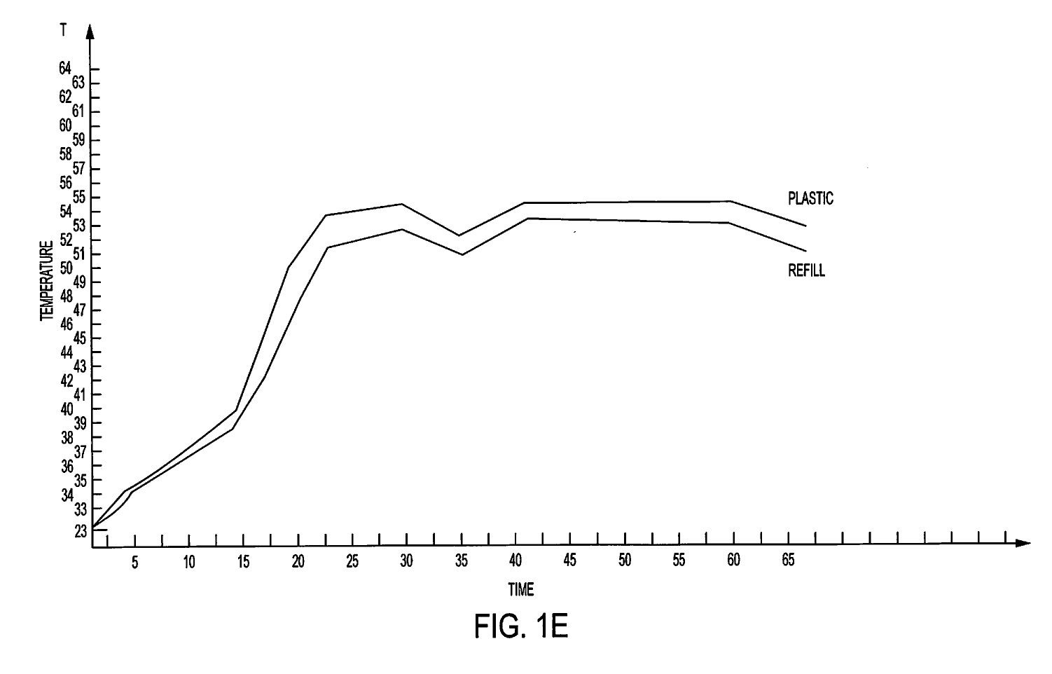

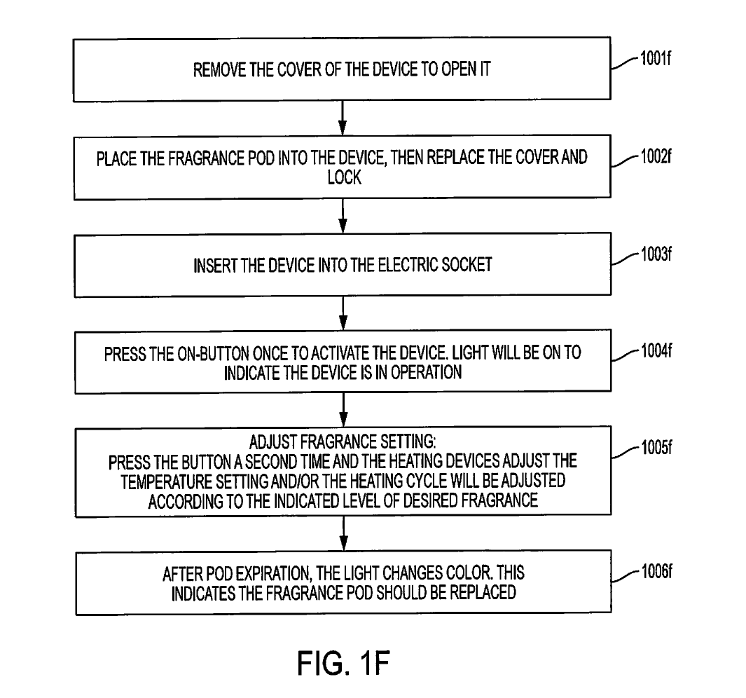

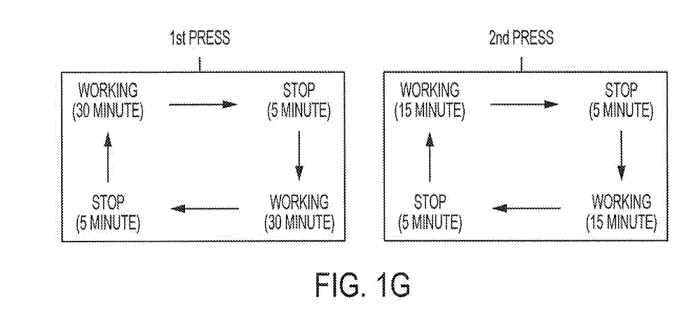

FIG. 1A demonstrates an example plug-in wickless fragrance device 100, which may be used according to any of the methods disclosed herein. Device 100 is shown from a top angle and bottom angle. Device 100 is also shown in a blown apart fashion from a top angle and a bottom angle. System 100 may include an exterior shell 101, which may serve as the body of the structure. Exterior shell 101 may be made of plastic, ceramic, glass, soft touch rubber, ceramic, glass, metal, silicone, wood, stone, synthetic or natural blends of various packaging materials. Exterior shell 101 may receive therein electrical contacts 102 (e.g., metal plug). Electrical contacts 102 may be compatible to be received by an electrical power source, such as, an electrical receptacle (e.g., wall socket, car cigarette lighter, AC input, DC input, USB input, light socket, cell phone input, headphones input, dongle, and the like. Exterior shell 101 also receives therein a printed circuit board (PCB) 103 having attached thereto a heating plate 103a. Heating plate 103a may include one or more heating devices hereon. Heating plate 103a may also include a switch and/or button 103b that may or may not comprise a light indicator. Exterior shell 101 may include a void 101a (e.g., hole) through which switch 103b may be access by a user.

Exterior shell 101 may also have receptacle 104 (e.g., holder) shaped and sized to receive a pod 105 (e.g., fragrance pod, substrate pod, etc.). Receptacle 104 may be made of plastic, ceramic, glass, soft touch rubber, ceramic, glass, metal, silicone, wood, stone, synthetic or natural blends of various packaging materials. Receptacle 104 may be positioned over PCB 103 within Exterior shell 101. Receptacle 104 may be shaped to include a tab 104a, which orients the position in which pod 105 is received. For example, if pod 105 is positioned within receptacle 104 in the wrong orientation, pod 105 will not rest properly due to tab 104a. If however, pod 105 is positioned within receptacle 104 in the correct orientation, pod 105 will rest properly due to tab 104a. Such an orientation may be used to control the way in which pod 105 is heated. For example, if pod 105 comprises multiple melting points and/or fragrances, its orientation in combination with the location of particular heating devices can be used to control the melting points and/or fragrances distributed therefrom.

Pod 105 may be a fragrance pod. Pod 105 is designed to release fragrance in light of a first driving force (e.g., passive diffusion). As such, pod 105 releases fragrance without the use of any energy (e.g., heat). In additional, pod 105 is designed to release fragrance in light of a second and different driving force (e.g., heat). Preferably, the second driving force is low heat. An example of temperatures utilized as the second driving force is shown in FIGS. 1E, 2G, and 4E. The addition of a second driving force, low heat, excites the aroma chemicals thereby pushing the aroma chemicals through the membrane more quickly and more efficiently than traditional passive diffusion membrane pods because pod 105 need not rely on a concentration gradient of aroma chemicals or added hydrocarbons to encourage movement of the aroma chemicals. As such, pod 105 creates a special upgraded functionality as compared to traditional membrane pods. Further, in contrast to typical heat driven pods, pod 105 does not melt.

Exterior shell 101 receives exterior lid 106, which is removeably connected thereto. Exterior lid 106 may snap, screw, clamp, magnetically connect and/or otherwise attach to exterior shell 101. In this example, magnets 106a hold lid 106 securely to exterior shell 101. Exterior lid 106 includes voids 106a (e.g., holes) threw which fragrance is able to escape. Voids 106a may be arranged in a predetermined pattern to maximize fragrance release and/or to enhance esthetics. Device 100 may include therein one or more light not shown. In examples, the light may be one or more LED, which displays one or more color (e.g., white, green, red, blue, yellow, etc.). The light may be used to communicate information to the user, as is described in further detail below. One or more light may illuminate through voids 106a. In such an embodiment, device 100 may function as a wickless fragrance diffuser and a lighting mechanism (e.g., night light, spot light, etc.).

FIG. 1B shows a profile view of device 100. FIG. 1B also shows some example dimensions, which may be the metric system or standard system. Of course any dimensions may be used as is desired. FIG. 1B also shows an example PCB 103a and heating plate 103a. FIG. 1C shows an example circuit diagram of PCB 103 and heating plate 103a.

FIG. 1D shows an example method of using a wickless fragrance device. In this example embodiment, at step 1001d, a user removes the cover of the exterior shell to open the device. In step 1002d, the user places a pod (e.g., fragrance pod) into the receptacle, and replaces the cover by setting the cover on the exterior shell and moving the cover into a locked position. In step 1003d, the user plugs the device into an electrical receptacle (e.g., wall socket). At step 1004d, the user presses an on-button to activate the device. If the device includes an indicator light, then upon activation of the device, a light is illuminated (e.g., green) indicating that the device is activated and operating correctly.

At step 1005d, one or more heating device on the heating plate heats to a temperature in the range of 40-50 degrees Celsius. One or more heating device may heat to 39, 40, 41, 42, 43, 44, 45, 46, 47, 48, 49, 50, or 51 degrees Celsius. The combined temperature of all the heating devices may be within one of the temperatures or the range mentioned above, while each specific heating device operates at a different heat from each other. Due to the design of the pod (described above), heating devices are operable to emit fragrance at lower temperatures as compared to other fragrance diffusers.

FIG. 1E illustrates an example heat cycle (e.g., heating plate) maintains the pod at optimum temperature & maximizes the fragrance delivery through the membrane. Device 100's ability to operate at a lower temperature provides improved safety as compared to traditional fragrance diffusers. Device 100 is less likely to burn the user or cause a fire. Further, device 100 operates with less energy thereby saving electricity and increasing efficiency.

Returning back to FIG. 1D, in step 1006d, one or more heating device may cycle. Humans (and animals) are susceptible to a condition called anosmia, which may be caused by an over stimulation of aroma chemicals on human's (or animal's) nasal receptors. The inventors hereof recognize the dangers posed by the oversaturation of fragrance in a particular space and have provided solutions to prevent this potential harmful side effect of traditional fragrance systems. For example, the systems described herein may cycle their aroma distributions. In embodiments, one or more heating device may cycle between being on and off and/or any temperature there between. In such an example, one or more heating device may be on for thirty minutes, off for five minutes, and then repeat. Of course the minutes of the cycles may be changed as is desired. In embodiments, a user may input information into the device indicting the desired length of time of each cycle.

Because a user has the ability to input information into the device indicting the desired length of time of each cycle, a user who is more or less prone to anosmia may control the device as is needed for their particular comfort. Further, a user may use personal preference regarding the cycle lengths and/or temperature selections that are desired for that particular user. In embodiments, pod 105 may be used to diffuse specific aromas, and a chosen cycle selection may be used to ensure preferred fragrance levels for the various specific aromas are released at proper time intervals.

While the user has the ability to input information into the device indicting the desired length of time of each cycle, some embodiments may include programming that prevent the user from inadvertently causing themselves anomia. For example, a wickless fragrance delivery device may comprise memory storing computer executable programming thereon, that when executed, helps to prevent human anosmia. In this embodiment, the wickless fragrance delivery device may receive input from a user defining a fragrance distribution cycle. Upon receiving the input, the circuitry of the wickless fragrance delivery may modify the user defined fragrance distribution cycle according to programming stored on memory therein. The modified fragrance distribution cycle may adjust (e.g., override, rewrite) the user defined fragrance distribution cycle to prevent the fragrance aroma chemicals from being distributed at a level that could potentially cause human anosmia for an average user or for that particular user.

At step 1007d, upon expiration of the fragrance pod, device 100 may turn off. In embodiments, the device may be programmed to turn off automatically and/or indicate to the user the desire to be turned off due to the fragrance pod's expiration. If device 100 is equipped with an indicator light, the light may communicate expiration of the fragrance pod, for example, by changing to a different color (e.g., from green to red). In embodiments, the light may flash in additional to or instead of changing color. The light change of step 1007d indicates to a user that the pod has expired and should be replaced.

In embodiments, the device tracks the time that the pod has been held within the device, and performs step 1007d upon expiration of a time limit (e.g., 5 hours, 30 days, and/or the like). In other embodiments, the device tracks an amount of time that the device has been activated since the pod has been held within the device, and performs step 1007d upon expiration of a time limit (e.g., 5 hours, 30 days, and/or the like). In other embodiments, the device has a fragrance analyzer, which determines an amount of fragrance being distributed from the pod, and performs step 1007d upon the amount of fragrance being distributed falling below a threshold.

FIG. 1F shows an example method of using device 100, wherein the user inputs a desired fragrance setting. In this example embodiment, at step 1001f, a user removes the cover of the exterior shell to open the device. In step 1002f, the user places a pod (e.g., fragrance pod) into the pod holder and replaces the cover by setting the cover on the exterior shell and moving the cover into a locked position. In step 1003f, the user plugs system 100 into an electrical receptacle (e.g., wall socket).

At step 1004f, the user provides input to the device indicating that the user desires the devices to release fragrance at a first programmed and/or programmable level (e.g., 100% intensity). Upon receiving input that the user desires a first programmable level (e.g., 100% fragrance intensity), the device illuminates one or more lights. A first light may indicate that the device is active. For example, a light may be illuminated as a particular color indicating activation (e.g., green). A second light may indicate the intensity of the fragrance being released. For example, a light may be illuminated at a particular intensity to indicate the intensity of the fragrance release (e.g., a light may be fully blue). In embodiments, more or less lights may be used as indication lights. For example, a single light may be illuminated at a particular color (e.g., green) and full intensity to indicate that the device is on and at 100% fragrance release (e.g., fully green light). Upon the device being activated, the heating devices of the heating plate are activated and heat up as is described above. In step 1004f, the user indicated a desire for a first programmable intensity (e.g., 100% fragrance intensity), so the heating devices will cycle according to a first timing schedule (e.g., 30 minutes on and 5 minutes off).

If the user desires the fragrance to be released at a different programmed and/or programmable intensity, such as, a second programmable intensity (e.g., less than 100% intensity), the user may move to step 1005f. In step 1005f, the user indicates a desire that the fragrance be released at a programmable intensity that is different from the first programmable intensity (e.g., less than 100% intensity) by inputting the desire into the device. For example, in step 1005f the user may press the button a second time. In embodiments, receiving a second input from the user indicates to the device that the user desires a second preprogrammed level of fragrance release. For example, the second input may indicate that the user desires a 50% fragrance intensity. In other examples, the device may accept several inputs which indicate several differences preprogrammed and/or programmable fragrance levels (e.g., ratios). For example, pressing the button once may indicate 100%, twice my indicate 90%, three times may indicate 80%, four times may indicate 70%, five times may indicate 60%, six times may indicate 50%, seven times may indicate 40%, eight times may indicate 30%, nine times may indicate 20%, and/or ten times may indicate 10%. In other examples, the indication may be made by holding the button down a predetermined amount of time. For example, holding the button down a first amount of time indicates a first percentage, holding the button down a second amount of time indicates a second percentage, and holding the button down a nth amount of time indicates a nth percentage (e.g., 1 second, 2 seconds . . . n seconds). Of course any indication may be used, for example twisting the button, inputting information from a smart phone application, speaking, clapping, etc.

In step 1005f, upon the device receiving input that the user desires a fragrance level that is different from the first setting, the device may adjust the timing of the heating devices' (and/or fans) cycles and/or the temperature of the heating devices. For example, the device may change one or more of the cycles' timing in order to increase or decrease the fragrance intensity. For instance, if the heating devices are set to cycle from 30 minutes on and 5 minutes off, the user may reduce the fragrance intensity by adjusting the heating devices to cycle from 24 minutes on and 8 minutes off. Additionally and/or alternatively, the user may reduce the fragrance intensity by adjusting the temperature of the heating devices while they are cycled on. Further, if the user wishes to increase the fragrance intensity, the user could set the device to cycle from 45 minutes on and 3 minutes off and/or adjust the temperature settings. Of course the times and temperatures mentioned above are merely examples. A user may input any cycle timing and/or temperature that is appropriate.

At step 1006f, upon expiration of the fragrance pod, one or more of the lights may change to a different color (e.g., from green to red). In embodiments, one or more of the lights may flash in additional to or instead of changing color. The light change of step 1006f indicates to a user that the pod has expired and should be replaced. Further, the device may automatically turn off the heating devices. In embodiments, the device tracks the time that the pod has been held within the device, and performs step 1006f upon expiration of a time limit (e.g., 5 hours, 30 days, and/or the like). In other embodiments, the device tracks an amount of time that the device has been activated since the pod has been held within the device, and performs step 1006f upon expiration of a time limit (e.g., 5 hours, 30 days, and/or the like). In this embodiment, if the user used the device at a lower fragrance intensity, the pod may last longer as compared to a user that used the device at a higher fragrance intensity. Intensity data may be used when determining whether the pod has expired. In other embodiments, the device has a fragrance analyzer, which determines an amount of fragrance being distributed from the pod, and performs step 1006f upon the amount of fragrance being distributed falling below a threshold.

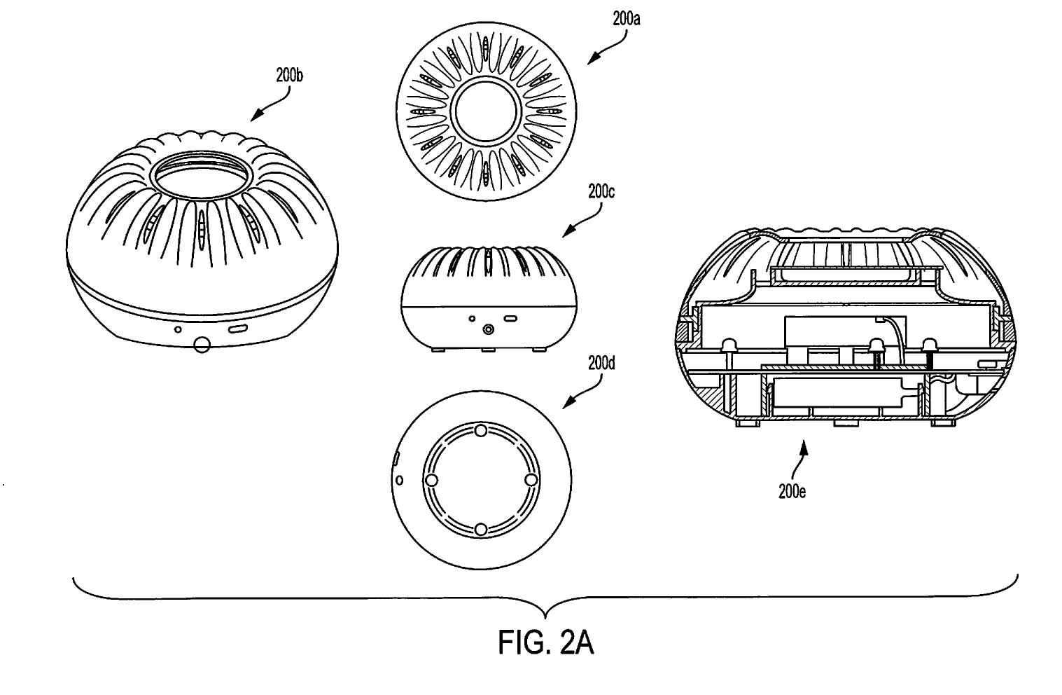

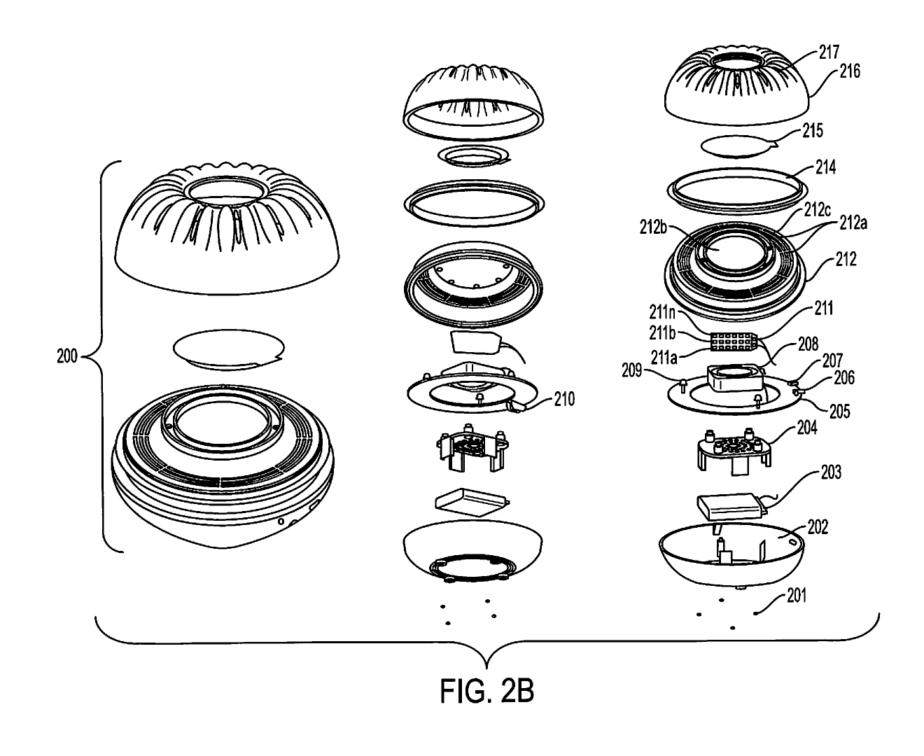

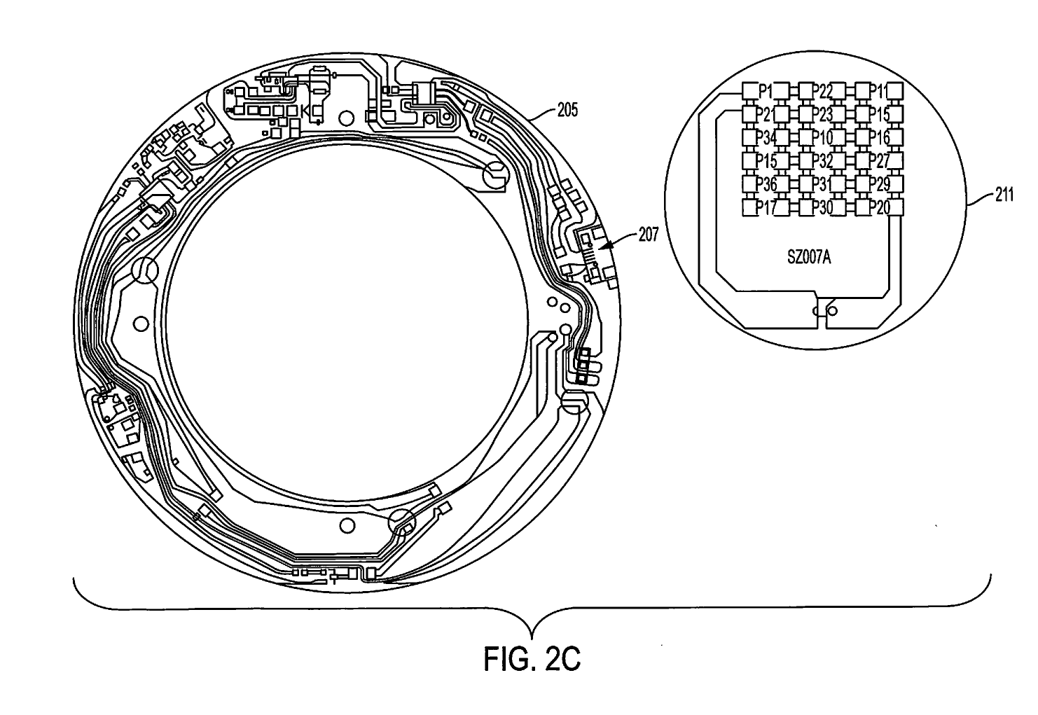

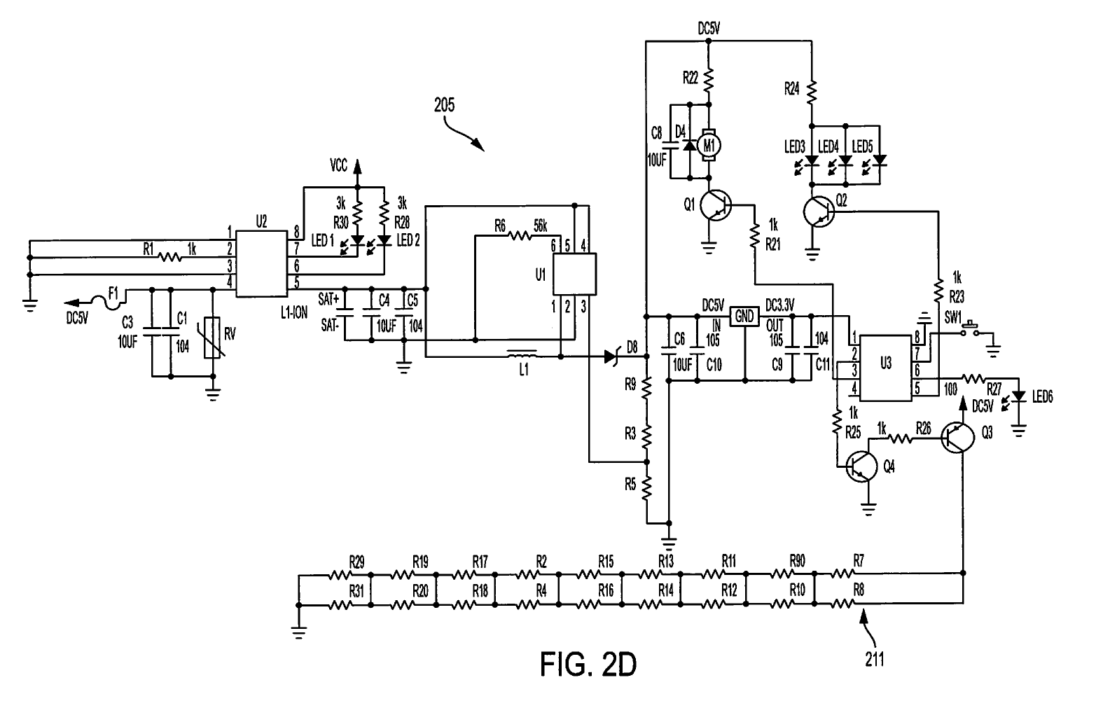

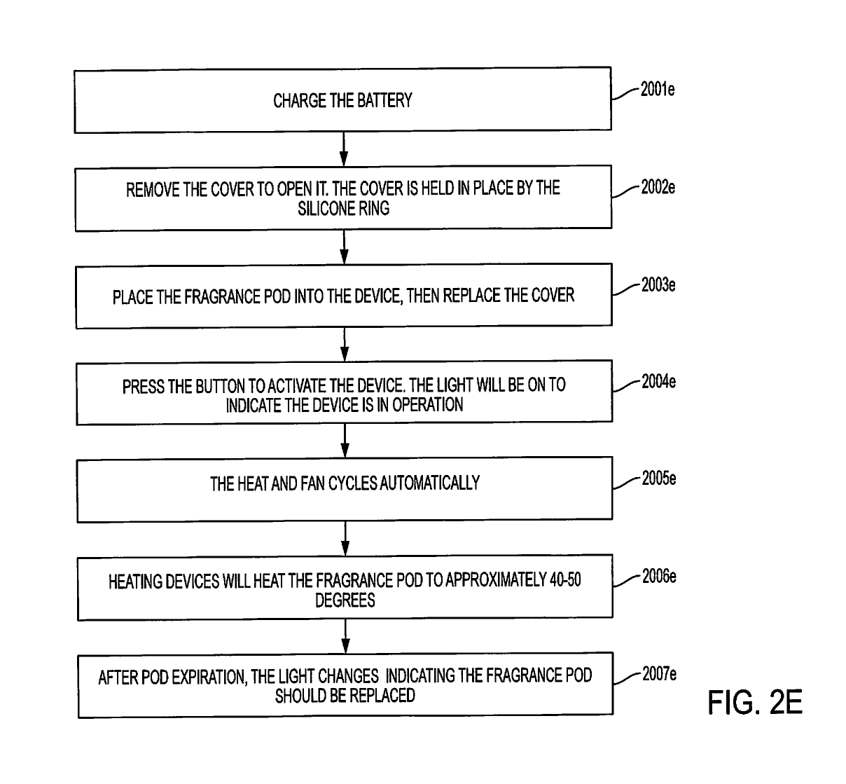

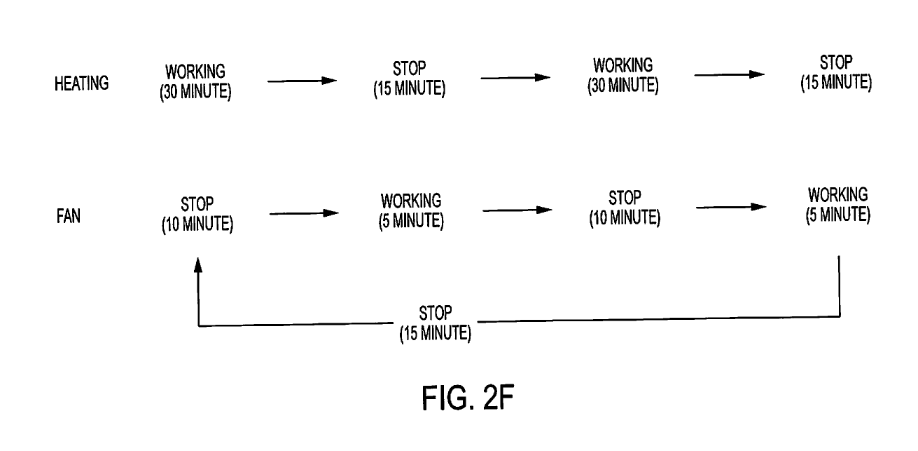

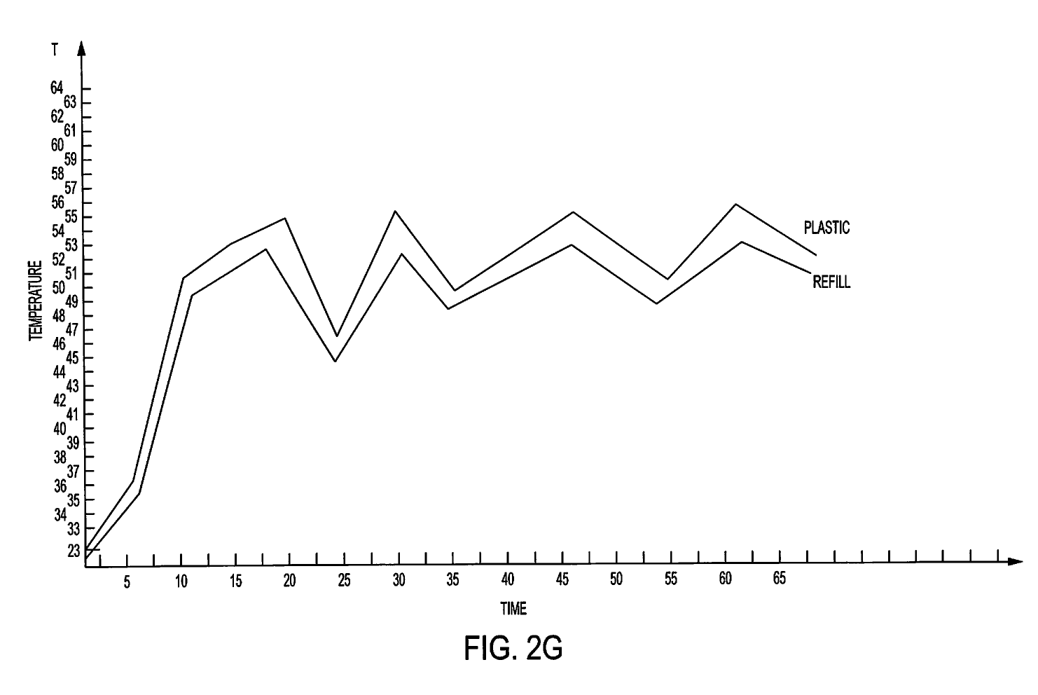

FIG. 2A demonstrates another wickless fragrance system 200, which may be used according to any of the methods disclosed herein. FIG. 2A shows a top view 200a, top angled view 200b, a profile view 200c, and a bottom view 200d. FIG. 2A also shows example dimensions 200e for device 200. Of course any dimensions may be used.

FIG. 2B shows top angled blown apart views of device 200 and a bottom angled blown apart view of device 200. Device 200 may include one or more pad 201 (e.g. silicon pads), which set between base 202 and the surface on which device 200 sits (e.g., a counter). One or more pad 201 are connected to the bottom side of base 202. Base may be made of plastic, ceramic, glass, soft touch rubber, ceramic, glass, metal, silicone, wood, stone, synthetic or natural blends of various packaging materials. Base 202 receives lithium battery 203, which may be rechargeable. Battery 203 may be recharged by being connected to a power source, for example, via a universal serial bus (USB) cable.

Device 200 may include a battery cover 204. In embodiments, battery 203 seats into base 202, and battery cover 204 seats into base 202 and covers battery 203. Battery cover 204 may be made of plastic, ceramic, glass, soft touch rubber, ceramic, glass, metal, silicone, wood, stone, synthetic or natural blends of various packaging materials. Resting on battery cover may be PCB 205. PCB 205 includes thereon one or more LEDs 209. LEDs 209 may be operable to provide ambient lighting. In this example, PCB 205 includes three LEDs. PCB 205 also includes switch and/or button 210. Switch 210 may operate as an on/off switch (or on/off button). PCB 205 may include one or more additional light 206 that is an indicator light. Indicator light 206 may be an LED. PCB 205 may also include at least one electrical and/or communication input/output 207. In this example, the input/output is a USB input/output. Base 202 may include holes through which indicator light 206, button 210, and input/output 207 are accessible and/or viewable by a user.

PCB 205 may rest on top of battery cover 204 and is powered by battery 203. Device 200 also includes one or more fan 208. In this embodiment, PCB 205 includes a cutout, and fan 208 rests within the cutout. Fan 208 is powered by battery 203 and programmably controlled by PCB 205. Device 200 also includes one or more heating device (211a-211n). In this example, heating devices (211a-211n) are located on heating plate 211. Heating devices (211a-211n) are powered by battery 203 and programmable controlled by PCB 205. In this embodiment, heating plate 211 is located above fan 208.

Device 200 also includes internal cover 212. Internal cover 212 may be made of plastic, ceramic, glass, soft touch rubber, ceramic, glass, metal, silicone, wood, stone, synthetic or natural blends of various packaging materials. Internal cover 212 may include voids 212a (e.g., venting holes). Internal cover 212 may include a receptacle 212b shaped and sized to receive a pod 215 (e.g., a membrane pod). Receptacle 212b may be a membrane pod holder and may be shaped to include a tab 212c, which orients the position in which pod 215 is received. Internal cover 212 may fit over (or otherwise couple to) base 202.

Device 200 may include ring 214. Ring 214 may be made of plastic, ceramic, glass, soft touch rubber, ceramic, glass, metal, silicone, wood, stone, synthetic or natural blends of various packaging materials. Silicon ring 214 slips over internal cover 212 and aids in securing lid 216 on internal cover 212. Lid 216 may include voids 217 (e.g., vents) allowing fragrance and/or light to escape from device 200.

FIG. 2C show example diagrams of PCB 205, heating plate 211, and USB port 207. USB port 207 is operable to be plugged into a power source in order to charge battery 203. Further, USB port 207 may be plugged into a computer and communicate computer executable programming instructions, which may be stored on a memory of PCB 205. FIG. 2D shows an example circuit diagram of one embodiment of PCB 205 and heating plate 211.

FIG. 2E illustrates an example method of using a wickless fragrance device. In step 2001e, a user may charge the battery of device 300. In embodiments, a light may communicate information to the user regarding the battery. For example, a light my illuminate a certain color to indicate the battery's current charge. For instance, the light may illuminate red indicting that the battery is in need of a charge. While the battery is charging, one or more light may indicate that charging is in process. For example, one or more light may illuminate a certain color (e.g., blue) indicating that charging is in process. Additionally and/or alternatively, the intensity and/or status of the light may indicate that the battery is charging. For example, the light may grow in illumination intensity and/or blink while charging is occurring. Further still, the light may change color, intensity, and/or status when charging is complete. For example, the light may turn green, turn on or off, increase or decrease in intensity, and/or blink or cease blinking as an indication that charging is complete. In some embodiments, it make take four hours to charge the battery.

In step 2002e, the user removes the cover to open the device. The cover may be held in place by a silicone ring. In step 2003e, the user places one or more fragrance pod into the device, and the cover is replaced. In step 2004e, the user provides input to the device indicating the user's desire that the device activate. The user may provide input by pressing a button. In embodiments, the button may be pressed one or more times to indicate the desired state of activation. Further, upon providing the input, the device may illuminate a light indicting the device is operational (e.g., heating the pod in order to distribute fragrance). The intensity and/or color of the light may indicate information to the user such as the operational state of the device and/or the remaining level of battery life of the device.

In step 2005e, a heating cycle will begin. In embodiments, a heating cycle will automatically turn one or more heating device on for 30 minutes at a time and turn the one or more heating devices off for 15 minutes at a time. The heating cycle may also include one or more fan, which cycles through on and off cycles. For example, during a heating cycle, a fan may be automatically activated for 10 minutes at a time and automatically deactivated for 5 minutes at a time. In embodiments, the timing of the cycles of the heating devices and the fans may be coordinated. For example, during the time period in which the heating devices are on, the fan may cycle one or more times (e.g., during a heating device's 30 minute on period, the fan is on for 10 minutes, off for 5 minutes, on for 10 minutes, and off for 5 minutes). Further, the fan may be coordinated such that the fan is off while the heating devices are off. Alternatively, the fan may be on while the heating devices are off. Any manner of coordinated cycles are conceived herein. An example of cycles are illustrated in FIG. 2F.

In step 2006e of FIG. 2E, while the one or more heating devices are on, the one or more heating device heats to a temperature as is described above. An example chart of temperature verses time is provided in FIG. 2G. At step 2007e of FIG. 2E, upon expiration of the fragrance pod, the device may automatically turn off and/or the light may change to a different color as is described above.

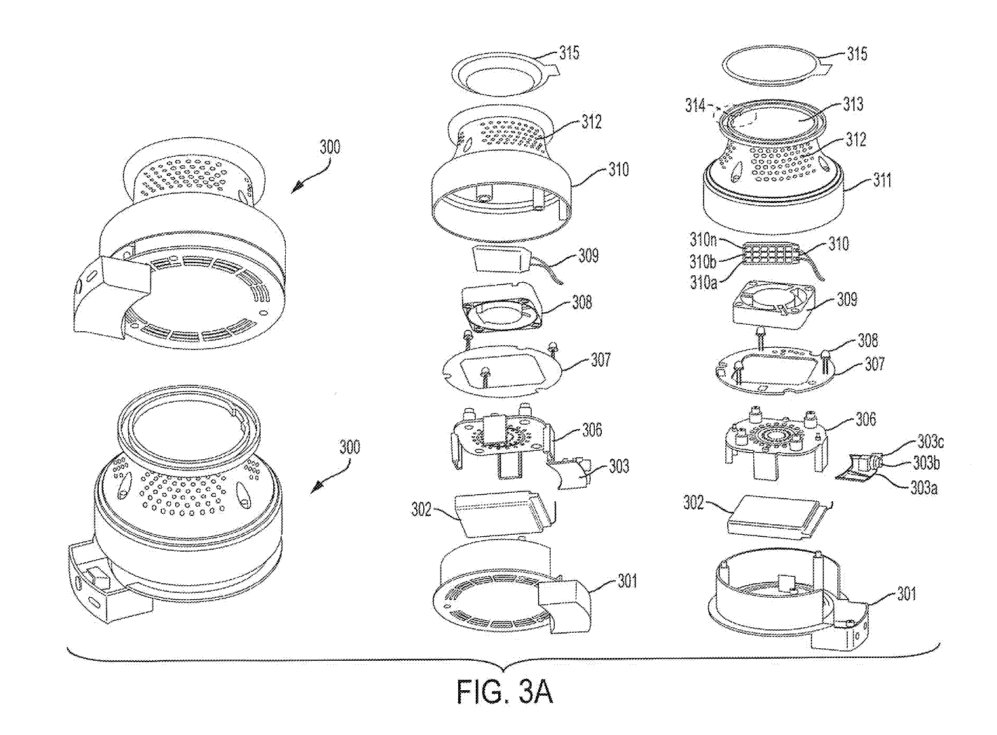

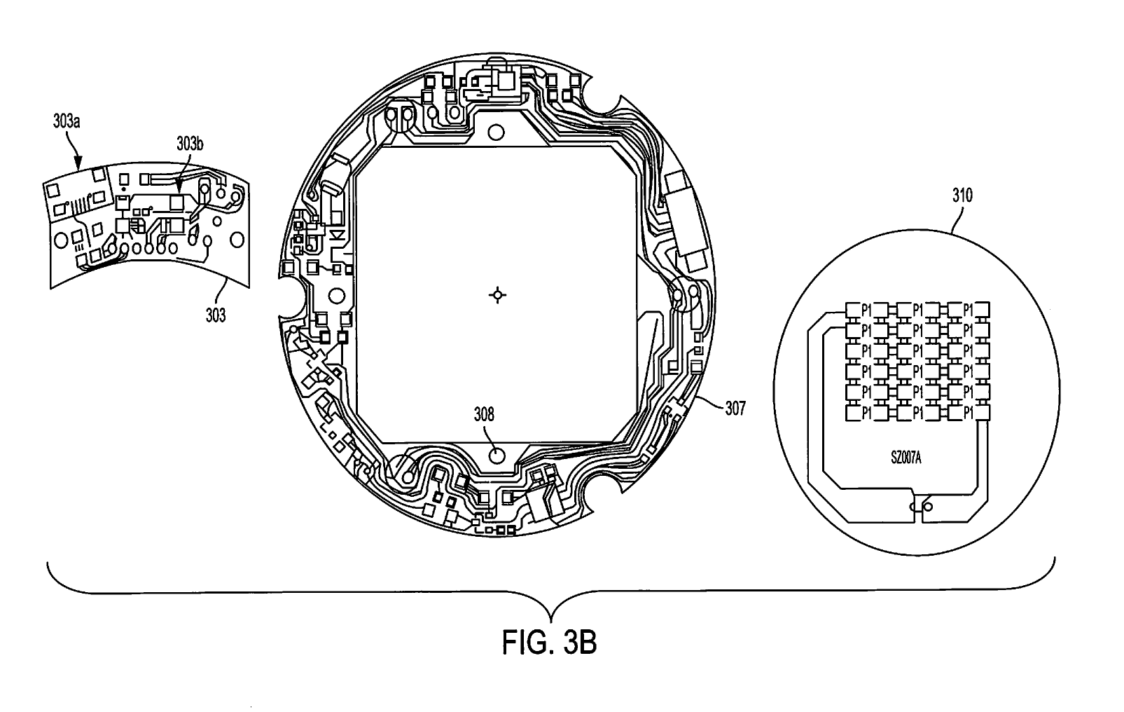

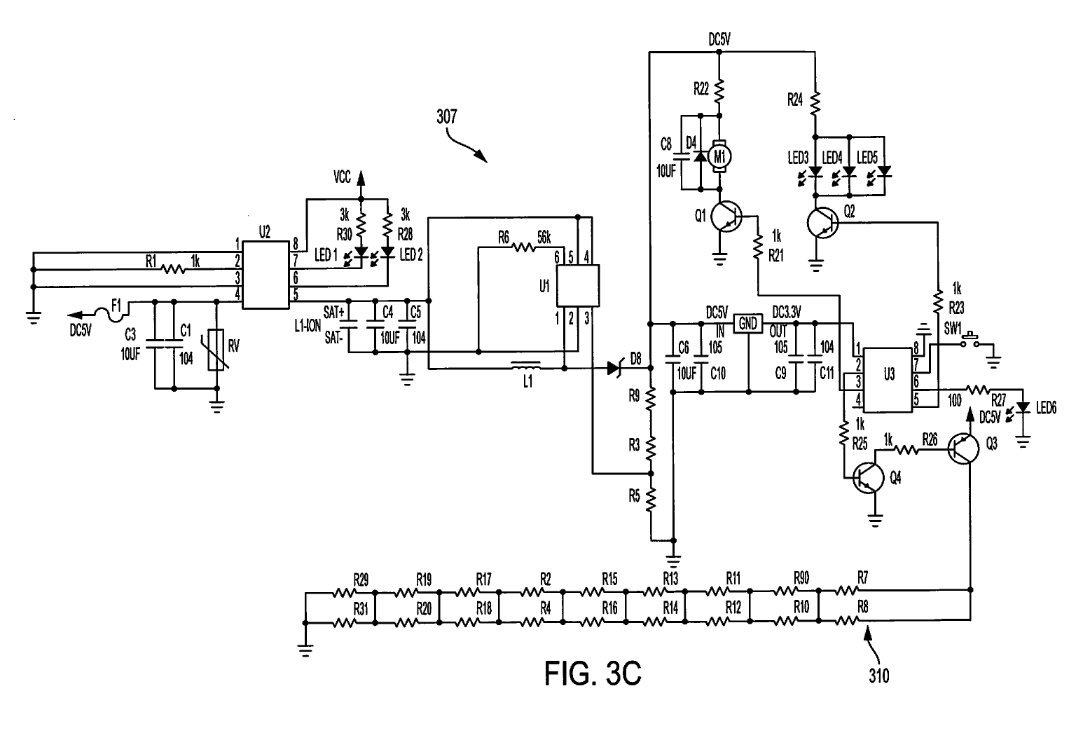

FIG. 3A demonstrates another wickless fragrance delivery device 300, which may be used according to any of the methods disclosed herein. In embodiments, device 300 may be housed within a housing, examples of which are described in more detail below. Further, any of the wickless fragrance delivery apparatuses described herein may be modified to include device 300. FIG. 3A shows device 300 from a top view and a bottom view in its assembled form. FIG. 3A also shows a bottom angle blown apart version of device 300 and a top angle blown apart version of device 300.

Device 300 includes a base 301. Base 301 may be made of plastic, ceramic, glass, soft touch rubber, ceramic, glass, metal, silicone, wood, stone, synthetic or natural blends of various packaging materials. Base 301 receives a battery 302 therein. In this example, battery 302 is similar to the battery described above. Battery 302 is connected to components of component plate 303. Upon component plate 303 is mounted at least one light 303a, at least one switch and/or button 303b, and at least one input/output 303c. Base 301 may include holes through which light 303a, button 303b, and input/output 303c are accessible and/or viewable by a user.

Device 300 may include a battery cover 306. In embodiments, battery 302 and component plate 303 seat into base 301, and battery cover 306 seats into base 301 and covers battery 302. Battery cover 306 may be made of plastic, ceramic, glass, soft touch rubber, ceramic, glass, metal, silicone, wood, stone, synthetic or natural blends of various packaging materials. Resting on battery cover may be PCB 307. PCB 307 includes thereon one or more LEDs 308, which may provide lighting (e.g., ambient lighting). In this example, PCB 307 includes three LEDs. PCB 307 may rest on top of battery cover 306 and is powered by battery 302. Device 300 also includes one or more fan 309. In this embodiment, PCB 307 includes a cutout, and fan 309 rests within the cutout. Fan 309 is powered by battery 302 and programmable controlled by PCB 307. Device 300 also includes one or more heating device (310a-310n). In this example, heating devices (310a-310n) are located on heating plate 310. Heating devices (310a-310n) are powered by battery 302 and programmable controlled by PCB 307. In this embodiment, heating plate 310 is located above fan 309.

Device 310 also includes cover 311. Cover 311 may be made of plastic, ceramic, glass, soft touch rubber, ceramic, glass, metal, silicone, wood, stone, synthetic or natural blends of various packaging materials. Cover 311 may include voids 312 (e.g., venting holes). Cover 311 may include a receptacle 313 shaped and sized to receive a pod 315 (e.g., a membrane pod). Receptacle 313 may be a membrane pod holder and may be shaped to include a tab 314, which orients the position in which pod 315 is received. Cover 311 may fit over (or otherwise couple to) base 301 in any manner discussed above. In embodiments, cover 311 may have holes that receive coupling mechanism, which couple cover 311 to base 301 (e.g., screw holes that receive screws).

FIG. 3B show example diagrams of PCB 307, heating plate 310, and component plate 303. FIG. 3C shows an example circuit diagram of one embodiment of PCB 307 and heating plate 310.

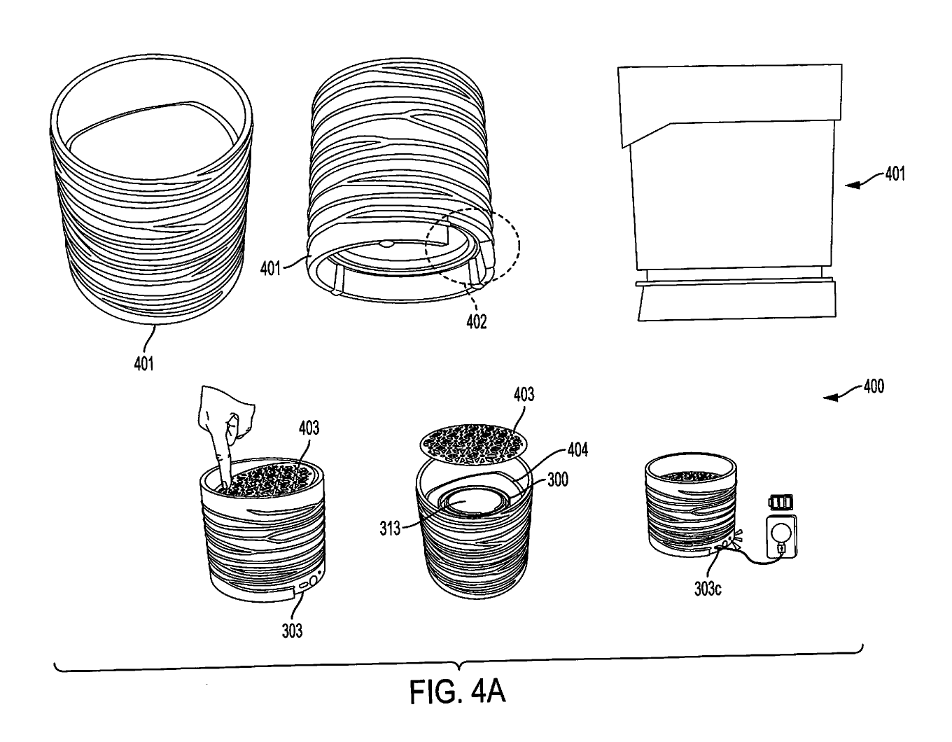

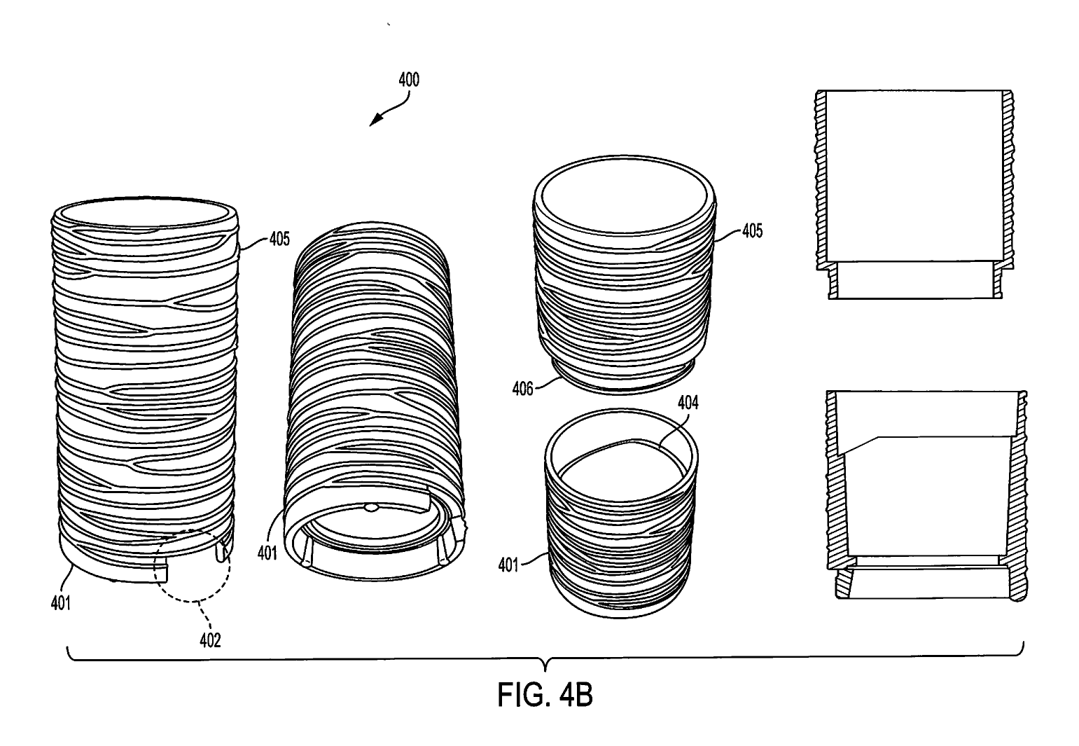

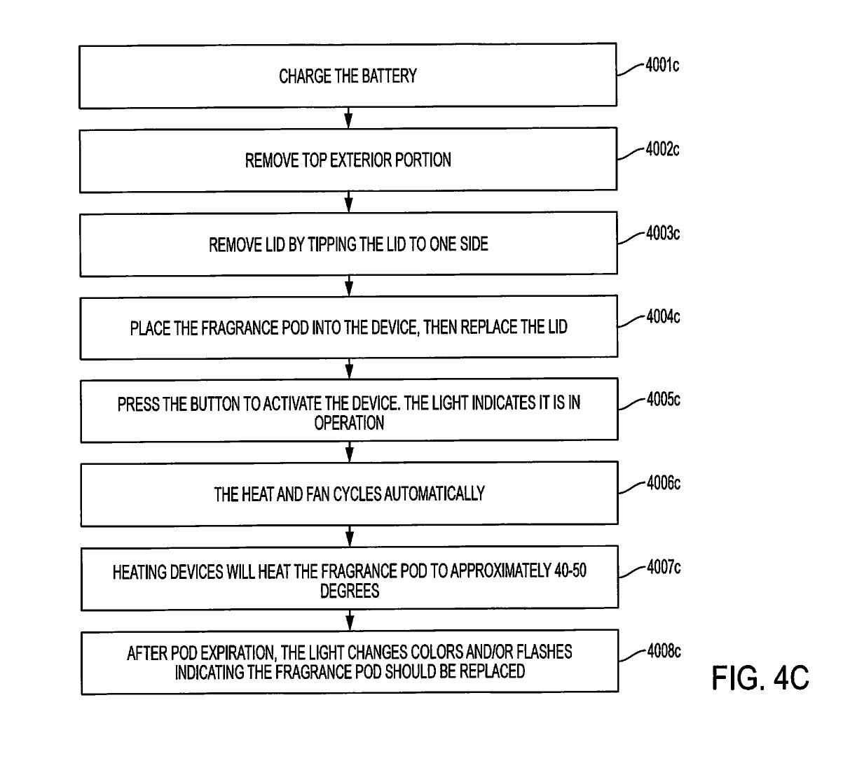

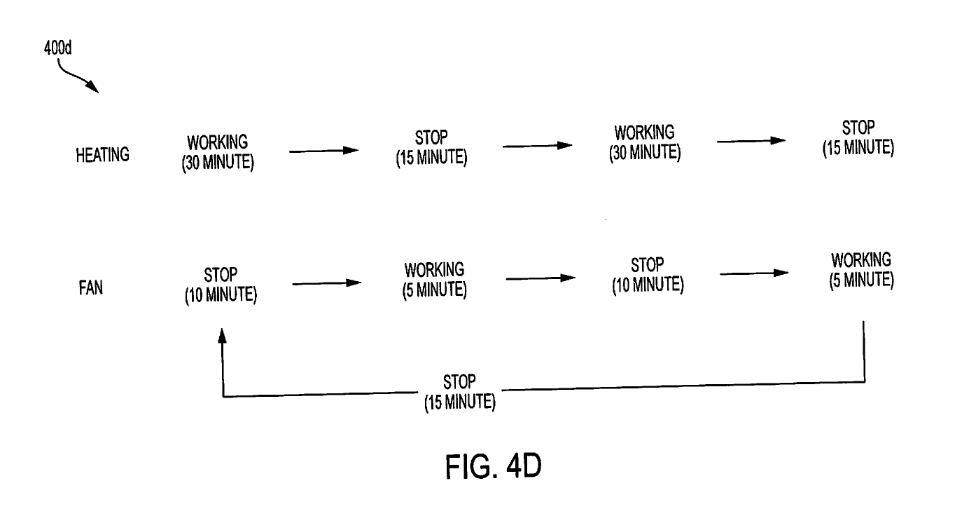

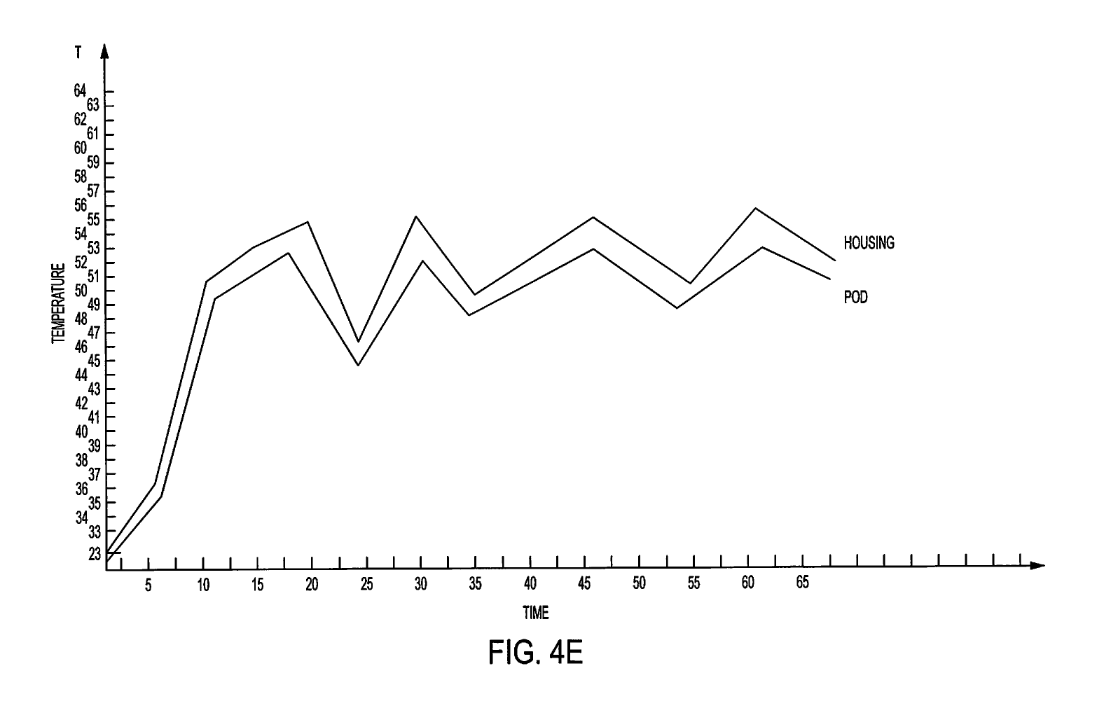

FIG. 4A shows an example housing 400, which may house a wickless fragrance delivery device (e.g., device 300), which may be used according to any of the methods disclosed herein. Housing 400 is shown from a top angle and a bottom angle. Housing 400 may be made of plastic, ceramic, glass, soft touch rubber, ceramic, glass, metal, silicone, wood, stone, synthetic or natural blends of various packaging materials. Housing 400 includes exterior portion 401, which may be designed to provide esthetics. Housing 400 may include a void 402 (e.g., cut out portion) shaped to provide user access to component plate 303. Housing 400 may be made of plastic, ceramic, glass, soft touch rubber, ceramic, glass, metal, silicone, wood, stone, synthetic or natural blends of various packaging materials. Lid 403 rests within the top portion of housing 400 and may be held in place by ledges 404. Ledges 404 are shaped such that lid 403 may be rocked, tilted, pushed down on one side by a user's finger. When lid 403 is tilted down on one side, the other side rises out of housing 400, and the user is able to grasp the lid 403 and remove it from housing 400 in order to place a pod into receptacle 313. In alternative embodiments, lid 403 may removable connect to housing 400 in any manner described herein. Lid 403 includes voids 403 (e.g., holes) threw which fragrance (e.g., from pod 315) and light (e.g., from LEDs 308) are emanate. Voids 403 may be arranged in a predetermined pattern to maximize fragrance release, light emission, and/or enhance esthetics. A user may charge the battery of device 300 by using a cord to connect electrical connector 303c and into a power source (e.g., wall socket, electrical outlet, battery charger, car socket, computer, etc.). FIG. 4A shows example dimensions for housing 400, but any dimensions may be used as is desired. In this example, a user may place housing 400, housing device 300 therein, on a table top, turn the device on, and enjoy fragrance. If device 300 includes lights (e.g., LEDs), the housing and device combination will appear to be a burning candle.

FIG. 4B shows another embodiment of a housing 4000 that may house device 300 or any wickless fragrance device. Housing 4000 is shown from a top angle and a bottom angle. Housing 4000 may include the components of housing 400 and add thereto. For example, housing 4000 may include top exterior portion 405 and bottom exterior portion 401. Bottom exterior portion may include void 402 through which component plate 303 is accessed and ledge 404 which supports lid 403. Top exterior portion may be removeably coupled to bottom exterior portion 401. In embodiments, top exterior portion 405 may include a lip 406, which inserts into bottom exterior portion 401 causing the top exterior portion 405 and bottom exterior portion 401 to appear to be a single unit. Top exterior portion 405 may be made of plastic, ceramic, glass, soft touch rubber, ceramic, glass, metal, silicone, wood, stone, synthetic or natural blends of various packaging materials. Top exterior portion 405 may be a different material as compared to bottom exterior portion 401. Bottom exterior portion 401 and/or top exterior portion 405 may be transparent, opaque, and/or nontransparent. In embodiments, bottom exterior portion 401 is nontransparent and top exterior portion 405 is opaque and/or transparent, such that when device 300 is activated, the combination emits fragrance from the pod and light from the LEDs, thus, appearing to be a lantern and/or candle. FIG. 4B shows example dimensions for housing 4000, but any dimensions may be used as is desired.

FIG. 4C illustrates an example method of using device 300 in conjunction with housing 400 and/or 4000. In step 4001c, a user may charge the battery of device 300. In embodiments, a light may communicate information to the user regarding the battery as is described above.

If device 300 is housed within housing 4000, then in step 4002c, the user removes the top exterior portion of the housing. If device 300 is housed within housing 400, then the user skips step 4002c. In step 4003c, the user removes the lid. In this example, the user removes the lid by tipping the lid to one side. Tipping the lid to one side causes the other side of the lid to lift above the housing. The user may gasp the portion of the lid lifting above the housing and remove the lid from the housing. In step 4004c, the user places a pod, e.g., fragrance pod, into the receptacle. The pod may include a tab which aligns with a tab inside the receptacle, thereby directing the orientation of the pod's placement within the housing. In step 4005c, the user activates the device. The user may activate the device by pressing a button. The device may indicate activation by illuminating a light as is described above.

In step 4006c, heating cycles and/or fan cycles may begin as are described above. An example of step 4006c is shown in FIG. 4D, which shows an example cycle 400d. ‘In step 4007c of FIG. 4C, while the one or more heating devices are on, the one or more heating devices heat to a temperature as is described above.

At step 4008c, upon expiration of the pod (e.g., when the pod no longer emits a sufficient amount of fragrance, leaks, becomes hazardous, and/or the like), the device may automatically turn off and/or a light may indicate the pod's expiration as is described above.

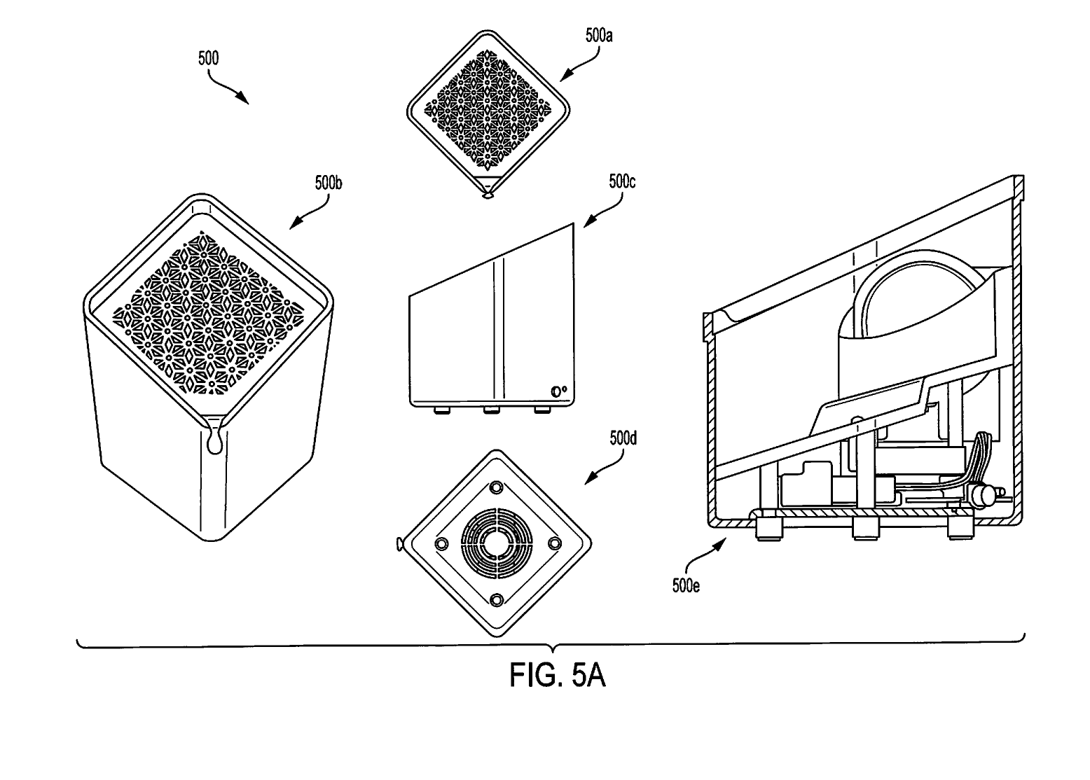

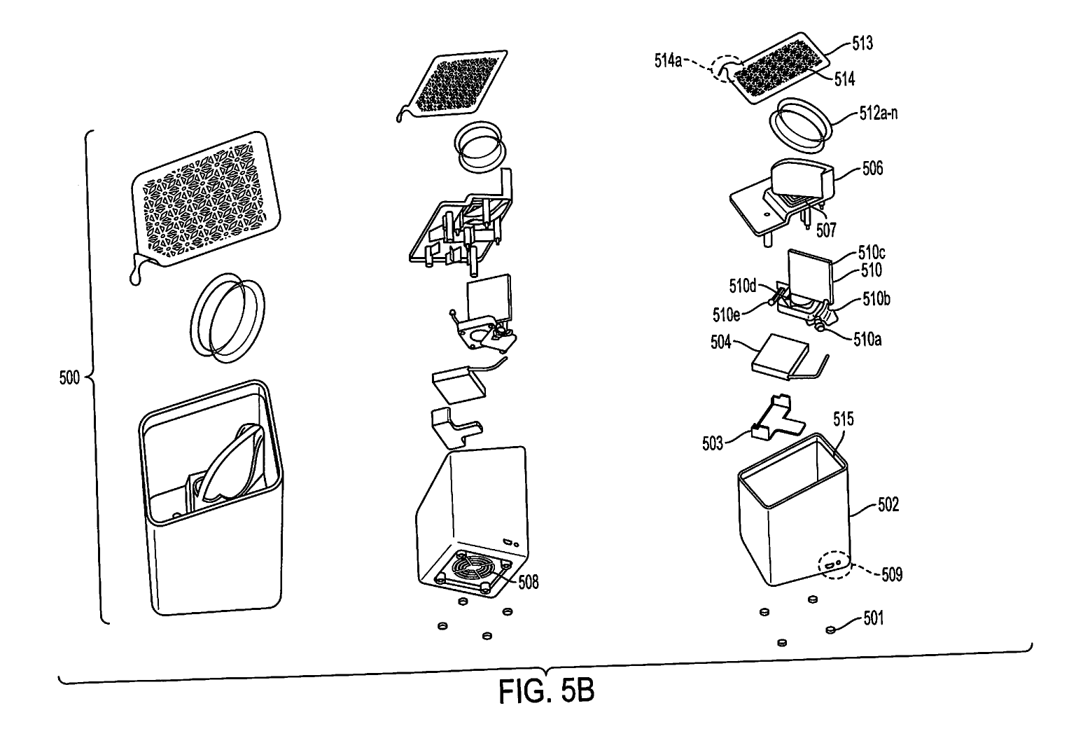

FIG. 5A shows another example wickless fragrance device 500, which may be used according to any of the methods disclosed herein. FIG. 5A shows a top view 500a, top angled view 500b, back view 500c, bottom view 500d, and example dimensions 500e of device 500. Of course any dimensions may be used for device 500.

FIG. 5B shows a top angled blown apart view of device 500 and a bottom angled blown apart view of device 500. Device 500 may include pads 501 on the bottom side of base 502. Pads 501 may be silicon, rubber, plastic, ceramic, glass, metal, and the like. Base 502 houses component parts of device 500 and may be made of plastic, ceramic, glass, soft touch rubber, ceramic, glass, metal, silicone, wood, stone, synthetic or natural blends of various packaging materials. Base may include voids 508 (e.g., vents), which distribute heat, fragrance, and/or light. Within base 502 is battery holder 503. Battery holder 503 may be made of plastic, ceramic, glass, soft touch rubber, ceramic, glass, metal, silicone, wood, stone, synthetic or natural blends of various packaging materials. Mounted with battery holder 503 is battery 504. Battery 504 may be similar to batteries described above.

Base 502 may also house component holder 510. Component holder 510 may include an on/off switch 510a, as is described above. Component holder 510 may also input/out 510b (e.g., USB), as is described above. Component holder 510 may include PCB 510c, as is described above. In embodiments, PCB 510c may exclude a centralized void and may not have one or more fan mounted within a centralized void. Component holder 510 may also include one or more fan 510d, as is described above. Component holder 510 many also include one or more lights 510e (e.g., LEDs), which may provide ambient lighting.

Base 502 may house pod holder 506. Pod holder 506 may be made of plastic, ceramic, glass, soft touch rubber, ceramic, glass, metal, silicone, wood, stone, synthetic or natural blends of various packaging materials. Pod holder 506 may include voids 507 (e.g., vents), which distribute heat, fragrance, and/or light. Pod holder may be shaped to receive one or more pods 512a-512n in a particular orientation, as is described above. Housing 500 may be made of plastic, ceramic, glass, soft touch rubber, ceramic, glass, metal, silicone, wood, stone, synthetic or natural blends of various packaging materials. Lid 514 may connect to housing 500 in any manner described herein. In embodiments, housing 500 includes ledge 515 upon which lid 514 rests. Lid 514 may also include clip 514a if desired, which may further secure lid 514 to housing 500.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A wickless fragrance delivery system comprising:
a power source;
a programmable printed circuit board (PCB) having memory thereon;
an input that receives input from a user selecting a programmable setting;
one or more heating device that is controlled by the PCB to transfer heat to one or more fragrance pod according to the selected programmable setting, wherein the selected programmable setting maintains a temperature between 40 and 55 degrees Celsius; and
the one or more wickless fragrance pod that emits a first amount of fragrance at room temperature and releases a second amount of fragrance between 40 and 55 degrees Celsius.

2. The system of claim 1 wherein the input further receives a second input from the user selecting a second programmable setting, wherein the one or more heating device is further controlled by the PCB according to the selected programmable setting.

3. The system of claim 1 wherein the one or more wickless fragrance pod comprises wax-free aroma chemicals.

4. The system of claim 1 wherein the one or more wickless fragrance pod comprises hydrocarbon-free aroma chemicals.

5. The system of claim 1 wherein the one or more wickless fragrance pod is sealed to prevent aroma chemicals contained therein from spilling out of one or more wickless fragrance pod while allowing gaseous particles to escape.

6. The system of claim 1 wherein the one or more heating device automatically turns off upon expiration of one or more wickless fragrance pod.

7. The system of claim 1 wherein the PCB automatically changes the selected programmable setting to a programmable setting that prevents aroma chemicals of the one or more fragrance pod from being distributed at a level that causes human anosmia.

8. The system of claim 1 further comprising:
one or more fan that is controlled by the PCB according the selected programmable setting.

9. The system of claim 8 wherein the one or more fan automatically turns off upon expiration of one or more wickless fragrance pod.

10. The system of claim 8 wherein the one or more heating device and the one or more fan is cyclically controlled by the PCB according to the selected programmable setting.

11. A method of delivering fragrance from a wickless fragrance delivery system comprising:
emitting, by one or more wickless fragrance pod, a first amount of fragrance at room temperature;
receiving input from a user selecting a programmable setting that is stored in a memory of a programmable printed circuit board (PCB);
controlling, the PCB according to the selected programmable setting, one or more one or more heating device that transfers heat to the one or more fragrance pod, wherein the selected programmable setting maintains a temperature between 40 and 55 degrees Celsius; and
releasing, by the one or more fragrance pod, a second amount of fragrance between 40 and 55 degrees Celsius.

12. The method of claim 11 further comprising:
receiving a second input from the user selecting a second programmable setting, wherein the one or more heating device is further controlled to heat the one more heating pod according to the selected programmable setting.

13. The method of claim 11 wherein the one or more wickless fragrance pod comprises wax-free aroma chemicals.

14. The method of claim 11 wherein the one or more wickless fragrance pod comprises hydrocarbon-free aroma chemicals.

15. The method of claim 11 wherein the one or more wickless fragrance pod is sealed to prevent aroma chemicals contained therein from spilling out of one or more wickless fragrance pod while allowing gaseous particles to escape.

16. The method of claim 11 wherein the one or more heating device automatically turns off upon expiration of one or more wickless fragrance pod.

17. The system of claim 11 further comprising:
changing, automatically by the PCB, the selected programmable setting to a programmable setting that prevents aroma chemicals of the one or more fragrance pod from being distributed at a level that causes human anosmia.

18. The method of claim 11 further comprising:
controlling one or more fan according the selected programmable setting.

19. The method of claim 18 wherein the one or more fan automatically turns off upon expiration of one or more wickless fragrance pod.

20. The method of claim 18 wherein the one or more heating device and the one or more fan is cyclically controlled by the PCB according to the selected programmable setting.
